(19) United States
(12) Patent Application Publication
(10) Pub. No.: US 2025/0099674 A1
(43) Pub. Date: Mar. 27, 2025

(54) ZERO GAP AUTOMATED INSULIN DELIVERY TREATMENT SYSTEM

(71) Applicant: Tandem Diabetes Care, Inc., San Diego, CA (US)

(72) Inventors: Geoffrey Kruse, San Diego, CA (US); Kia MirSanaei, Poway, CA (US); Hetal Babla, San Diego, CA (US); Sabah Mufti, San Diego, CA (US); Michelle Booke, San Diego, CA (US); Lori Cahill, McHenry, IL (US)

(21) Appl. No.: 18/474,839

(22) Filed: Sep. 26, 2023

(51) Int. Cl. A61M 5/142

(52) U.S. Cl. CPC A61M 5/14248; A61M 2202/0486; A61M 2205/50

(57) ABSTRACT

Disclosed herein are systems and methods for transitioning between different CGM sensors in an infusion pump system with no loss in continuity of glucose levels between different sensor for use in closed loop diabetes therapy. Notifications can be provided to the user when a subsequent CGM sensor ought to be inserted, based on specific mechanics of the previous CGM sensor and of the anticipated subsequent CGM sensor. The system can further provide a notification when the subsequent CGM sensor is activated following the warm up period and capable of transmitting CGM data for use in the system and can prompt the user to or automatically transition the system from communicating with the previous sensor for CGM data to the subsequent sensor. Following the transition, the system may also notify the user that the previous CGM sensor can be removed from the user's body.

### TECHNICAL FIELD

The present disclosure is directed generally to systems and methods relating to portable infusion pumps and more particularly to continuous glucose monitoring in infusion pump systems.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type 1, or in some cases, type 2 diabetes. Some insulin injecting pumps are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Ambulatory infusion pumps have generally been controlled by a user interface provided on the pump. With the proliferation of handheld electronic devices, such as mobile phones (e.g., smartphones), there is a desire to be able to remotely utilize such devices, as well as dedicated wireless controllers designed to work with one or more infusion pumps and/or types of infusion pumps, to optimize usage of infusion pumps. These remote controllers would enable a pump to be monitored, programmed and/or operated more privately, more conveniently and more comfortably. Accordingly, one potential use of dedicated remote devices and handheld consumer electronic devices (such as smartphones, tablets and the like) is to utilize such devices as controllers for remotely programming and/or operating infusion pumps.

Conventionally, portable infusion pump systems have been limited by the ability of corresponding sensors. For example, a continuous glucose monitoring (CGM) sensor generally has a “warmup period,” between insertion of the CGM sensor into interstitial tissue of a user and active use of the CGM sensors, where CGM data is not available or detected by the CGM sensor. During this period, users do not have access to CGM readings, and therefore the portable infusion pumps are unable to provide delivery of insulin in conjunction with closed loop continuous glucose monitoring.

### SUMMARY

Disclosed herein are systems and methods for transitioning between different CGM sensors in an infusion pump system with no loss in continuity of glucose levels between different sensors for use in closed loop diabetes therapy. Notifications can be provided to the user when a subsequent CGM sensor ought to be inserted, based on specific mechanics of the previous CGM sensor and of the anticipated subsequent CGM sensor. The system can further provide a notification when the subsequent CGM sensor is activated following the warm up period and capable of transmitting CGM data for use in the system and can prompt the user to or automatically transition the system from communicating with the previous sensor for CGM data to the subsequent sensor. Following the transition, the system may also notify the user that the previous CGM sensor can be removed from the user's body.

In an embodiment, an ambulatory infusion pump system can include a pump mechanism configured to deliver insulin to a user, a communications interface configured to receive data indicative of glucose levels of a user from one or more continuous glucose monitoring sensors and at least one processor configured to receive the data indicative of glucose levels of the user and to determine therapy parameters for the user based on the glucose levels. The at least one processor can be configured to determine that an ongoing active use state of a first CGM sensor will be expiring. A notification can be provided to the user to insert a second CGM sensor a predetermined amount of time prior to expiration of the first CGM sensor upon determining that the first CGM sensor will be expiring.

In an embodiment, a method of diabetes therapy delivering insulin to a user based on glucose levels of a user received from a first CGM sensor. If it is determined that an ongoing active use state of the first CGM sensor will be expiring a notification can be provided to the user to insert a second CGM sensor a predetermined amount of time prior to expiration of the first CGM sensor.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 depicts an embodiment of a pump system according to the disclosure.

FIG. 2 depicts a block diagram representing an embodiment of a pump system according to the disclosure.

FIGS. 3A-3B depicts an embodiment of a pump system according to the disclosure.

FIG. 4 depicts an embodiment of a pump system according to the disclosure.

FIGS. 5A-5B depict remote control devices for a pump system according to embodiments of the disclosure.

FIG. 6 depicts a schematic representation of a pump system according to an embodiment of the disclosure.

FIG. 7 depicts a flowchart of methods steps for monitoring CGM session active use and determining optimal insertion time for a subsequent CGM in connection to an infusion pump system according to an embodiment of the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

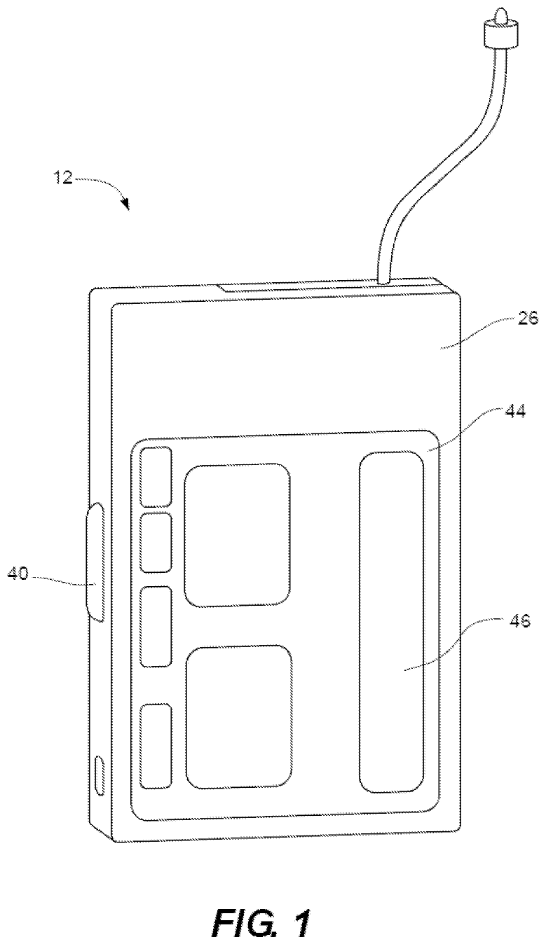

FIG. 1 depicts an exemplary medical device that can be used with embodiments of the disclosure. In this embodiment, the medical device is configured as a pump 12, such as an infusion pump, that can include a pumping or delivery mechanism and reservoir for delivering medicament to a patient and an output/display 44. The type of output/display 44 may vary as may be useful for a particular application. The output/display 44 may include an interactive and/or touch sensitive screen 46 having an input device such as, for example, a touch screen comprising a capacitive screen or a resistive screen. The pump 12 may additionally include a keyboard, microphone, or other input device known in the art for data entry, which may be separate from the display. The pump 12 may also include a capability to operatively couple to one or more blood glucose meters (BGMs) or continuous blood glucose monitors (CGMs) and/or one or more secondary devices such as a remote display, a remote control device, a laptop computer, personal computer, tablet computer, a mobile communication device such as a smartphone, a wearable electronic watch, smart ring, electronic health or fitness monitor, or personal digital assistant (PDA), a CGM display etc.

In one embodiment, the medical device can be a portable pump configured to deliver insulin to a patient. Further details regarding such pump devices can be found in U.S. Pat. No. 8,287,495, which is incorporated herein by reference in its entirety. In other embodiments, the medical device can be an infusion pump configured to deliver one or more additional or other medicaments to a patient.

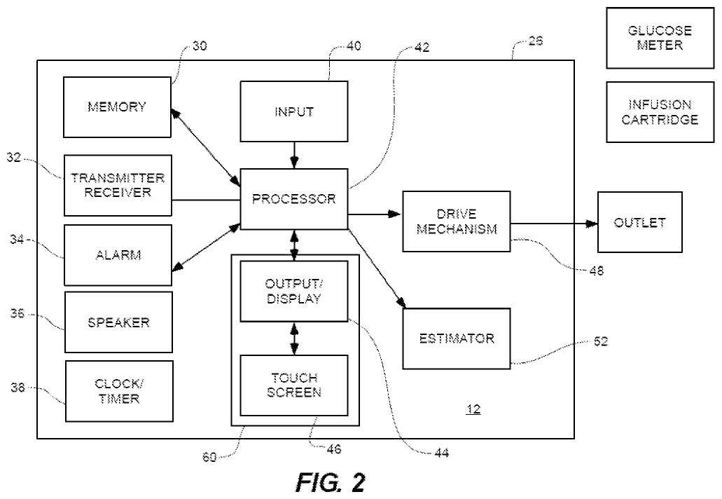

FIG. 2 illustrates a block diagram of some of the features that can be used with embodiments, including features that may be incorporated within the housing 26 of a medical device such as a pump 12. The pump 12 can include a processor 42 that controls the overall functions of the device. The infusion pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface as shown in FIG. 2 is a graphical user interface (GUI) 60 having a touch sensitive screen 46 with input capability. In some embodiments, the processor 42 may communicate with one or more other processors within the pump 12 and/or one or more processors of other devices, for example, a continuous glucose monitor (CGM), display device, smartphone, etc. through the transmitter/receiver. The processor 42 may also include programming that may allow the processor to receive signals and/or other data from one or more input devices, such as sensors that may sense pressure, temperature and/or other parameters.

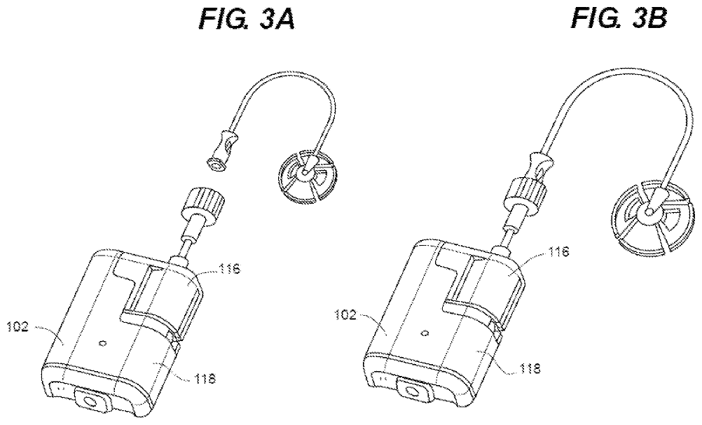

FIGS. 3A-3B depict a second infusion pump that can be used in conjunction with one or more embodiments of the ambulatory infusion pump system of the present disclosure. Pump 102 includes a pump drive unit 118 and a medicament cartridge 116. Pump 102 includes a processor that may communicate with one or more processors within the pump 102 and/or one or more processors of other devices such as a remote device (e.g., a CGM device), a remote control device, or a consumer electronic device (e.g., laptop computer, personal computer, tablet computer, smartphone, electronic watch, smart ring, electronic health or fitness monitor, or personal digital assistant). The processor 42 may also include programming to receive signals and/or other data from an input device, such as, by way of example, a pressure sensor, a temperature sensor, or the like. Pump 102 also includes a processor that controls some or all of the operations of the pump. In some embodiments, pump 102 receives commands from a separate device for control of some or all of the operations of the pump. Such separate device can include, for example, a dedicated remote control device or a consumer electronic device such as a smartphone having a processor executing an application configured to enable the device to transmit operating commands to the processor of pump 102. In some embodiments, processor can also transmit information to one or more separate devices, such as information pertaining to device parameters, alarms, reminders, pump status, etc. Such separate device can include any remote display, remote control device, or a consumer electronic device as described above. Pump 102 can also incorporate any or all of the features described with respect to pump 12 in FIG. 2. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. Further details regarding such pumps can be found in U.S. Pat. No. 10,279,106 and U.S. Patent Publication Nos. 2016/0339172 and 2017/0049957, each of which is hereby incorporated herein by reference in its entirety.

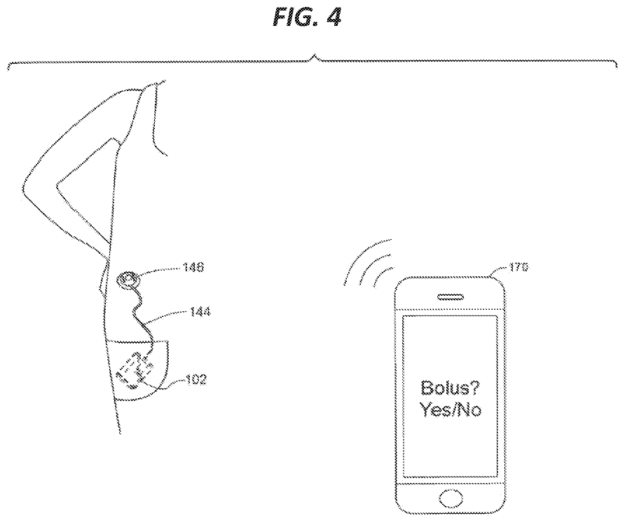

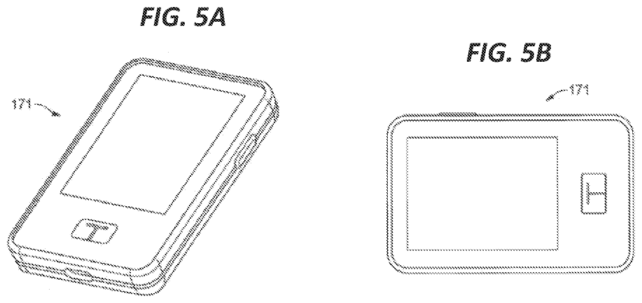

Referring to FIGS. 4-5B, one or more remote control devices 170, 171 can be used to communicate with the processor of pump 12 and/or pump 102 to control delivery of medicament and transfer data with pump via a wired or a wireless electromagnetic signal, such as via, e.g., a near field communication (NFC) radio frequency (RF) modality or other RF modalities such as Bluetooth®, Bluetooth® low energy, mobile or Wi-Fi communication protocols, for example, according to embodiments of the present disclosure. Such a remote control can include, for example, a mobile communication device 170, such as a smart phone (as depicted in FIG. 4) executing a software application for control of the pump, a dedicated remote controller 171 (as depicted in FIGS. 5A-5B), a wearable electronic watch, smart ring, electronic health or fitness monitor or personal digital assistant (PDA), etc., or a tablet, laptop or personal computer. Such communications between (and among) the one or more remote control devices 170, 171 and pump 102 may be one-way or two-way for, e.g., effective transfer of data among the devices and the pump, control of pump operations, updating software on the devices and/or pump, and allowing pump-related data to be viewed on the devices and/or pump.

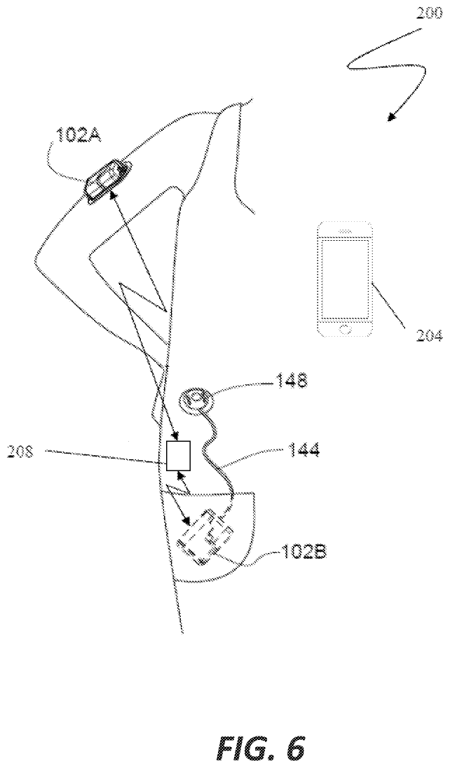

FIG. 6 depicts a schematic representation of a pump system 200 according to an embodiment. System 200 includes a user-wearable infusion pump such as pump 12 or pump 102 described above. In embodiments, a user can alternatively wear the pump 102A directly on the body or place the pump 102B in the user's pocket or other location near the body with infusion tubing 144 extending to an infusion set 148 on the user's body. The system 200 also includes a continuous glucose monitoring (CGM) sensor with a corresponding transmitter 208. The CGM sensor obtains measurements relating to glucose levels in the body and the transmitter communicates that information to the pump 102A/B. The pump 208 can then use the glucose data in making therapy determinations. The system can also include a one or more devices such as a smartphone 204 or other multi-purpose consumer electronic device capable of operating a software application to communicate with and/or control the pump and, alternatively or additionally, a dedicated remote control device designed specifically for use with pump 102A/102B. The smartphone 204 or other remote control device can in some embodiments also be capable of communication with CGM sensor/transmitter 208. In addition, the pump 102A/B and/or smartphone 204 or other remote control device can optionally communicate with additional devices such as, for example, a blood glucose meter or other analyte sensing device, an activity or other health monitor, etc.

Although depicted with the multi-purpose consumer electronic device 204 being a smartphone, in various embodiments the consumer electronic device can alternatively or additional include one or more of a wearable electronic watch, such as a smartwatch, a smart ring, electronic health or fitness monitor, personal digital assistant (PDA), or a tablet, laptop or personal computer, etc. A multi-purpose consumer electronic device can be any device sold to consumers and used for a variety of functions and which can be configured or programmed to communicate with and/or control an infusion pump as one of said functions. In some embodiments, systems as described herein may include more than one multi-purpose consumer electronic device configured for communication with the infusion pump (e.g., a smartphone and a smart watch).

It should be noted that although embodiments herein are primarily described with routing of communications from a CGM, that the communication methodologies disclosed herein can relate to other devices in infusion pump systems that can have communications with a pump or other device in the system. For example, the disclosure can relate to additional devices such as, for example, another analyte sensing device, an activity or other health monitor, heart rate monitor, etc.

During an active CGM session, the CGM sensor will continually measure glucose levels of the user for use in therapy calculations in the system. However, CGM sessions are only activated after a period of time after a CGM sensor is inserted into the body of the user, which can be referred to as a “warm up” period. This warm up period (the period between insertion of a CGM sensor into interstitial tissue of the user and activation of a CGM session) is necessary because CGM sensors comprise porous material which requires wetting with interstitial tissue in order to absorb glucose and accurately monitor glucose levels. CGM readings are therefore unavailable to monitor the glucose levels of the user during this warm up period. The warm up period for CGM sensors can typically range anywhere from 30 minutes to 2 hours. As such, during the warm up period, the system is unable to provide automated delivery of insulin in conjunction with closed loop continuous glucose monitoring, as the system is also unable to access CGM data before activation of a CGM session.

One potential solution to this issue is for users to insert a subsequent CGM sensor before a previous CGM session has ended. In such a scenario, the user must be responsible for correctly estimating the end time of the previous CGM session and adequately anticipating the warm up period necessary for the subsequent CGM sensor to become active. Various issues can occur with manual management of CGM sessions in this manner, including the user remembering when to insert the next CGM sensor, knowing when the sensor is warmed up and transitioning from the old sensor to the new sensor prior to expiration of the old sensor. Timing misalignment may be difficult to avoid through manual management, not in the least because different types of CGM sensors have different warm up periods.

Therefore, it would be beneficial to provide an infusion pump system that provides notifications to the user when a subsequent CGM sensor ought to be inserted, based on specific mechanics of the previous CGM sensor and of the anticipated subsequent CGM sensor. The system can further provide a notification when the subsequent CGM sensor is activated following the warm up period and capable of transmitting CGM data for use in the system and can prompt the user to or automatically transition the system from communicating with the previous sensor for CGM data to the subsequent sensor. Following the transition, the system may also notify the user that the previous CGM sensor can be removed from the user's body. Such notifications and messages can be provided to the user by one or more devices in such a system including, for example, the pump, a mobile communication device, such as a smart phone executing a software application for control of the pump, a dedicated remote controller, a wearable electronic watch, smart ring, electronic health or fitness monitor or personal digital assistant (PDA), etc., or a tablet, laptop or personal computer.

The system can therefore be configured to track various information such as CGM session cycle state, anticipated CGM session end time, and optimal insertion time of subsequent CGM sensor. In some embodiments, the CGM sensor can communicate to the system when the sensor is set to expire in a predetermined amount of time. Alternatively or additionally, the system can track anticipated expiration of a CGM sensor based on known characteristics of the type of sensor. Display messages intended to notify the user can be displayed in increments (e.g., 1 hour before optimal insertion time, 30 minutes before optimal insertion time, etc.) and can be modified by the user. Display messages can also be programmed to occur during preferred times (e.g., in the evening before a Do Not Disturb status is set, etc.).

In some embodiments of such a system, the pump can be configured to connect to both the previous CGM sensor and the subsequently inserted CGM sensor concurrently as the subsequent CGM sensor is in its warm up period. Modification of preexisting Bluetooth settings on the pump and/or remote devices may be necessary in order for the pump to effectively master both the previous CGM sensor and the subsequent CGM sensor. In embodiments, even as dual connections from the previous and subsequent CGM sensors to the pump emerge, monitored glucose level readings will only be used from one sensor (for example, during the warm up period of the subsequent sensor, only readings from the previous sensor shall be used). As the CGM cycle for the previous CGM sensor ends and as the warm up period for the subsequent CGM sensor ends, the pump may automatically disconnect from the previous CGM sensor and connect to the new sensor. As such, closed loop insulin therapy can continue without interruption even though the user has changed CGM sensors. Just as device (or the pump) can display a prompt to the user to install a new sensor, similarly, after the warm up period of the new subsequent CGM sensor has completed, a prompt may be displayed urging the user to remove the expired previous CGM sensor.

In some embodiments, the CGM sensors may be configured to transmit messages to a remote device or to the pump to display to the user that the sensor has completed its warm up or that it is on its way to expiration. In other embodiments, the pump or other device may interrogate the CGM sensor at regular interval to determine the CGM status. While it is typical for users to be aware of how long a CGM sensor generally lasts before expiration (e.g., 10 days, etc.), the user can be provided with notification reminders regarding typical expiration timelines.

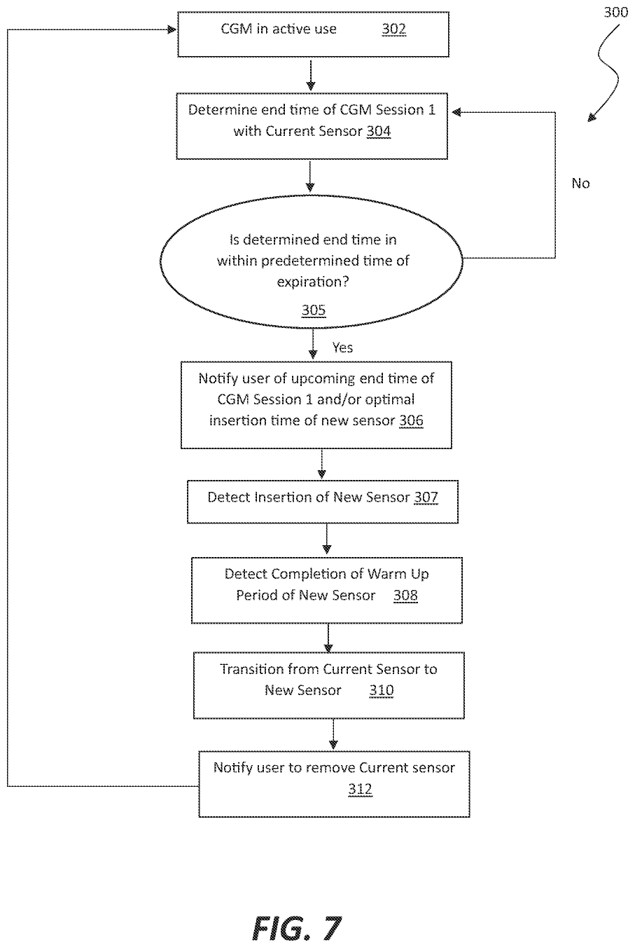

Referring now to FIG. 7, a flowchart of method steps for a method 300 of monitoring active use state of a CGM session is depicted. At step 302, a current CGM sensor is operably and/or communicatively coupled to one or more remote control devices and/or a pump and is in an active use state such that the current CGM sensor corresponds to an actively ongoing CGM session that will continually measure glucose levels of the user for use in therapy calculations in the system. Generally, predetermined duration of active use of a CGM session corresponds to the type of CGM sensor, where each type of CGM sensor indicates a different duration of active use.

At step 304, processor of the pump and/or processing circuitry of the remote control devices may be configured to determine whether the session of active use for the CGM is still ongoing and, based on the type of CGM, determine an anticipated end of the active use session for the CGM. Specifically, at step 304, the method 300 may be configured to determine the anticipated end time of an ongoing CGM session based in part on the CGM type.

At step 305, a check can occur to determine whether the anticipated end time determined in step 304 is within the predetermined time of expiration processed in step 302. If the anticipated end time determined in step 304 does not fall within the predetermined time of expiration as processed in 302, the method 300 can revert to step 304 in which the CGM is in active use and the system generally monitors for upcoming expiration of the current sensor. If the anticipated end time determined in step 304 falls within the predetermined time of expiration as processed in step 302, the method 300 can be configured to proceed to step 306.

In some embodiments, the system will maintain the first CGM session until it is determined at step 305 the CGM session will end within a predetermined amount of time, which may be based on known characteristics of the type of CGM sensor. In other embodiments, the determination at step 305 may occur based on a communication sent from the CGM sensor to the system of an upcoming anticipated expiration of the sensor.

At step 306, notifications can be provided to the user alerting the user of the upcoming expiration and/or indicating an optimal time to insert the new CGM on one or more display screens of the pump, remote control device or other connected device. Notification messages can be provided to inform the user when the active CGM session is anticipated to end. The notification messages can be preset or may be configured to user preferences (e.g., 30 minutes before end of an active session, before a Do Not Disturb setting is scheduled to begin, etc.). The system may further prompt the user of an optimal time to insert a new CGM sensor, either at the same time the user is notified of the upcoming expiration of the previous sensor, at the optimal time, or both. In some embodiments, the system may prompt the user when to insert a new CGM sensor based on the characteristics of the previous CGM sensor. In other embodiments, the user may be prompted to or otherwise enter a type of CGM sensor that will be inserted, and the optimal time to insert the new sensor based on the entered type of sensor.

At step 307, the method 300 can be configured to detect the insertion of a new CGM sensor, where the new CGM sensor can be inserted within the predetermined time of expiration of the current CGM session and before the anticipated end of the CGM session as determined in step 304.

At step 308, the method 300 is configured to detect the termination of the warm up period of the new inserted CGM sensor. Warm up periods may vary based on type of CGM. Differing CGM types may indicate varying warm up periods. For example, a certain type of CGM may have a warm up period around 30 minutes, where the inserted CGM is not in active use and unable to provide monitoring of blood glucose levels or to provide continuous closed loop delivery of insulin, as needed, based on the monitored blood glucose levels. In other types of CGMs, the warm up period may be as long as 2 hours. Therefore, at step 308, detection of termination of the warm up period is crucial, as warm up periods may greatly vary. This determination may be based, for example, on the known characteristics of the CGM, monitoring of the data detected by the CGM for accuracy, etc.

At 310, after warm up of the new CGM sensor has been completed, data from the previous (current) sensor session will be transitioned to the new CGM sensor session. For example, data detected by the previous (current) sensor may be transferred to the new CGM sensor session based on the data received by the pump, remote control device, etc.

At 312, the method 300 will be configured to display notifications available to the user, for example on remote control device, pump, etc. to indicate that the previous (current) CGM sensor can be removed because it is no longer in use.

In an embodiment, an ambulatory infusion pump system can include a pump mechanism configured to deliver insulin to a user, a communications interface configured to receive data indicative of glucose levels of a user from one or more continuous glucose monitoring sensors and at least one processor configured to receive the data indicative of glucose levels of the user and to determine therapy parameters for the user based on the glucose levels. The at least one processor can be configured to determine that an ongoing active use state of a first CGM sensor will be expiring. A notification can be provided to the user to insert a second CGM sensor a predetermined amount of time prior to expiration of the first CGM sensor upon determining that the first CGM sensor will be expiring.

In some embodiments, the at least one processor is configured to determine that the first CGM sensor will be expiring based on characteristics of a type of CGM sensor of the first CGM sensor.

In some embodiments, the at least one processor is configured to determine that the first CGM sensor will be expiring based on a communication received from the first CGM sensor.

In some embodiments, the predetermined amount of time is a predetermined amount stored in a memory.

In some embodiments, the predetermined amount of time is based on one or more characteristics of a type of CGM sensor of the first CGM sensor.

In some embodiments, the one or more characteristics includes a warmup up period of time required upon insertion of the type of CGM sensor required before data provided by the type of CGM sensor can be used for determining therapy parameters.

In some embodiments, the predetermined amount of time is sufficient to enable the warmup of period of time to be completed prior to expiration of the first CGM sensor.

In some embodiments, the at least one processor is further configured to monitor a warmup up period of time required upon insertion of the second CGM sensor required before data provided by the type of CGM sensor can be used for determining therapy parameters.

In some embodiments, the at least one processor is configured to automatically transition from using data from the first CGM sensor to using data from the second CGM sensor for calculating therapy parameters upon expiration of the warmup period.

In some embodiments, the at least one processor is configured to provide a notification to the user to remove the first CGM sensor from the user's body following transitioning to the second CGM sensor.

In an embodiment, a method of diabetes therapy delivering insulin to a user based on glucose levels of a user received from a first CGM sensor. If it is determined that an ongoing active use state of the first CGM sensor will be expiring a notification can be provided to the user to insert a second CGM sensor a predetermined amount of time prior to expiration of the first CGM sensor.

In some embodiments, determining that the first CGM sensor will be expiring is based on characteristics of a type of CGM sensor of the first CGM sensor.

In some embodiments, the method further includes receiving a communication from the first CGM sensor that it will be expiring and determining that the first CGM sensor will be expiring is based on the communication.

In some embodiments, the predetermined amount of time is a predetermined amount stored in a memory.

In some embodiments, the method further includes determining the predetermined amount of time based on one or more characteristics of a type of CGM sensor of the first CGM sensor.

In some embodiments, the one or more characteristics includes a warmup up period of time required upon insertion of the type of CGM sensor required before data provided by the type of CGM sensor can be used for determining therapy parameters.

In some embodiments, the predetermined amount of time is sufficient to enable the warmup of period of time to be completed prior to expiration of the first CGM sensor.

In some embodiments, the method further includes monitoring a warmup up period of time required upon insertion of the second CGM sensor required before data provided by the type of CGM sensor can be used for determining therapy parameters.

In some embodiments, the method further includes automatically transitioning from using data from the first CGM sensor to using data from the second CGM sensor for calculating therapy parameters upon expiration of the warmup period.

In some embodiments, the method further includes providing a notification to the user to remove the first CGM sensor from the user's body following transitioning to the second CGM sensor.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492, 141; 10/541,987; 10,569,016; 10,736,037; 10,888,655; 10,994,077; 11,116,901; 11,224,693; 11,291,763; 11,305,057; 11,458,246; 11,464,908; and 11,654,236 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2021/0353857; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545 and 2023/0113755 and commonly owned U.S. patent application Ser. Nos. 17/368,968; 17/896,492; 18/011,060; 18/071,814; 18/071,835; 18/075,029; 18/090,788 18/115,316; and Ser. No. 18/139,391.

For purposes of interpreting the claims, it is expressly intended that the provisions of 35 U.S.C. § 112 (f) are not to be invoked unless the specific terms “means for” or “step for” are recited in a claim.

1. An ambulatory infusion pump system, comprising:
a pump mechanism configured to deliver insulin to a user;
a communications interface configured to receive data indicative of glucose levels of a user from one or more continuous glucose monitoring sensors; and
at least one processor configured to receive the data indicative of glucose levels of the user and to determine therapy parameters for the user based on the glucose levels, the at least one processor configured to:
determine that an ongoing active use state of a first CGM sensor will be expiring; and
provide a notification to the user to insert a second CGM sensor a predetermined amount of time prior to expiration of the first CGM sensor upon determining that the first CGM sensor will be expiring.

2. The ambulatory infusion pump system of claim 1, wherein the at least one processor is configured to determine that the first CGM sensor will be expiring based on characteristics of a type of CGM sensor of the first CGM sensor.

3. The ambulatory infusion pump system of claim 1, wherein the at least one processor is configured to determine that the first CGM sensor will be expiring based on a communication received from the first CGM sensor.

1. An ambulatory infusion pump system, comprising:
a pump mechanism configured to deliver insulin to a user;
a communications interface configured to receive data indicative of glucose levels of a user from one or more continuous glucose monitoring sensors; and
at least one processor configured to receive the data indicative of glucose levels of the user and to determine therapy parameters for the user based on the glucose levels, the at least one processor configured to:
determine that an ongoing active use state of a first CGM sensor will be expiring; and
provide a notification to the user to insert a second CGM sensor a predetermined amount of time prior to expiration of the first CGM sensor upon determining that the first CGM sensor will be expiring.

2. The ambulatory infusion pump system of claim 1, wherein the at least one processor is configured to determine that the first CGM sensor will be expiring based on characteristics of a type of CGM sensor of the first CGM sensor.

3. The ambulatory infusion pump system of claim 1, wherein the at least one processor is configured to determine that the first CGM sensor will be expiring based on a communication received from the first CGM sensor.

4. The ambulatory infusion pump system of claim 1, wherein the predetermined amount of time is a predetermined amount stored in a memory.

5. The ambulatory infusion pump system of claim 1, wherein the predetermined amount of time is based on one or more characteristics of a type of CGM sensor of the first CGM sensor.

6. The ambulatory infusion pump system of claim 5, wherein the one or more characteristics includes a warmup up period of time required upon insertion of the type of CGM sensor required before data provided by the type of CGM sensor can be used for determining therapy parameters.

7. The ambulatory infusion pump system of claim 6, wherein the predetermined amount of time is sufficient to enable the warmup of period of time to be completed prior to expiration of the first CGM sensor.

8. The ambulatory infusion pump system of claim 1, wherein the at least one processor is further configured to monitor a warmup up period of time required upon insertion of the second CGM sensor required before data provided by the type of CGM sensor can be used for determining therapy parameters.

9. The ambulatory infusion pump system of claim 8, wherein the at least one processor is configured to automatically transition from using data from the first CGM sensor to using data from the second CGM sensor for calculating therapy parameters upon expiration of the warmup period.

10. The ambulatory infusion pump system of claim 9, wherein the at least one processor is configured to provide a notification to the user to remove the first CGM sensor from the user's body following transitioning to the second CGM sensor.

11. A method of diabetes therapy, comprising:
delivering insulin to a user based on glucose levels of a user received from a first CGM sensor;
determining that an ongoing active use state of the first CGM sensor will be expiring; and
providing a notification to the user to insert a second CGM sensor a predetermined amount of time prior to expiration of the first CGM sensor upon determining that the first CGM sensor will be expiring.

12. The method of claim 11, wherein determining that the first CGM sensor will be expiring is based on characteristics of a type of CGM sensor of the first CGM sensor.

13. The method of claim 11, further comprising receiving a communication from the first CGM sensor that it will be expiring and wherein determining that the first CGM sensor will be expiring is based on the communication.

14. The ambulatory infusion pump system of claim 11, wherein the predetermined amount of time is a predetermined amount stored in a memory.

15. The ambulatory infusion pump system of claim 11, further comprising determining the predetermined amount of time based on one or more characteristics of a type of CGM sensor of the first CGM sensor.

16. The method of claim 15, wherein the one or more characteristics includes a warmup up period of time required upon insertion of the type of CGM sensor required before data provided by the type of CGM sensor can be used for determining therapy parameters.

17. The method of claim 16, wherein the predetermined amount of time is sufficient to enable the warmup of period of time to be completed prior to expiration of the first CGM sensor.

18. The method of claim 11, further comprising monitoring a warmup up period of time required upon insertion of the second CGM sensor required before data provided by the type of CGM sensor can be used for determining therapy parameters.

19. The method of claim 18, further comprising automatically transitioning from using data from the first CGM sensor to using data from the second CGM sensor for calculating therapy parameters upon expiration of the warmup period.

20. The method of claim 19, further comprising providing a notification to the user to remove the first CGM sensor from the user's body following transitioning to the second CGM sensor.
